(19) United States
(12) Patent Application Publication
(10) Pub. No.: US 2016/0262340 A1
(43) Pub. Date: Sep. 15, 2016

(54) Method and System for Cleaning and Sanitising Agroindustrial Farms

(71) Applicant: QUÍMICA ROSMAR S.A. DE C.V., Iztapalapa, México, D.F. (MX)

(72) Inventors: Rodrigo Martínez Romero, Mexico City (MX); Humberto Martínez Romero, Mexico City (MX)

(21) Appl. No.: 15/034,190

(22) PCT Filed: Dec. 19, 2013
(86) PCT No.: PCT/MX2013/000195

§ 371 Date: May 4, 2016

(51) Int. Cl. A01K 1/01; B08B 3/02; B01F 3/04; B01F 15/02; B05B 12/08; A01K 41/00; A61L 2/18; F04B 17/05; F04C 15/00; B05B 7/00; B08B 3/00; A01K 31/04

(52) U.S. Cl. CPC A01K 1/01; B08B 3/003; B08B 3/02; B01F 3/04446; B01F 15/0216; A01K 31/04; A01K 41/00; A61L 2/18; F04B 17/05; F04C 15/008; B05B 7/0025; B05B 12/08; B01F 2215/004

(57) ABSTRACT

The present invention relates to a process for cleaning and sanitizing productive agribusiness areas such as production farms, reproduction farms, poultry, pig and egg incubators, using a system that includes a standalone mobile unit which includes a foam-producing unit for applying a specialized detergent formulation for use with livestock and a pressure washing unit for pre-rinsing and rinsing, as well as to the cleaning and sanitizing process using the system that includes the standalone unit, making it possible to increase cleaning efficiency and productivity, and to reduce the death rate among poultry and pigs for reasons related to ineffective sanitation.

### FIELD OF THE INVENTION

The present invention lies in the field of cleaning and sanitation systems. Specifically, the present invention relates to a process for cleaning and sanitization of agribusiness productive areas such as production farms, breeding farms, incubators for birds, pigs, and eggs, with a system that includes an autonomous mobile unit comprising a foam producing unit to apply a specialized detergent formulation for livestock, and a pressure water unit for pre-rinse and rinse; and the process for cleaning and sanitization, achieving increased cleaning productivity and lower mortality of birds and pigs for reasons inherent in ineffective sanitization.

### BACKGROUND OF THE INVENTION

For cleaning in different situations it has been found and documented that having a foam generating equipment facilitates the cleaning process so that it is better and more effective. One of the fields where it is necessary to have high standards of cleanliness to ensure biosafety, is livestock and agricultural, where little has been innovated on this matter in recent years.

Biosafety assurance and decreasing mortality and morbidity in agricultural production is not limited to the proper use of vaccines and medicines, or the appropriate selection of efficient processes and practices in terms of farm operation to guarantee the same. The first step to health quality assurance is to incorporate in the operative practices the concept that the cleaning and sanitizing process should provide a clean working environment, and also an equipment in good sanitary conditions, all this aimed to the prevention of pollution and disease control, microbiologically monitored.

This assurance of sanitary quality leads to a decrease in drugs and medicines and to abatement of losses due to mortality, which now, in some cases, reach up to 20-25% mortality per cycle; and thus the losses are very high for sharecroppers and Companies.

In terms of productivity, in a large number of cases are being done at most 5 out of 6 to 7 production cycles per year, so in order to not extend the non-production cycles, sanitation is poor and thus the mortality rises. In other cases with high mortality and poor sanitation, the waiting period for repopulation is increased, further reducing the cycles.

Also, contamination by cages and trucks is very high with regional health risks, without overlooking that microbiology usually is not measured, and where is measured is very high; also, there is no certainty of being free of pathogenic viruses (e.g. influenza H7N3).

In addition to this, agribusiness farms face high costs in disinfectants due to poor sanitation and a waste of time resulting in increased costs, since at present, a team of workers can clean 4,500 m2 per day, which represents 30% less than acceptable global standard.

Patent U.S. Pat. No. 7,959,091 discloses a mobile cleaning unit that produces foam, the unit is useful for cleaning and sanitizing work areas. However, said document does not disclose large-capacity foam generation, since to date prior art equipment for generating foam do poorly because of the complexity in the technology to make a foam mixture rich and dry and its movement for dosing it at high speed and in less time. Also derived that the unit is designed for controlled operations within an industrial plant with controlled utilities (electricity, water, air and areas), and relatively small dimensions.

One of the problems that arise in agribusiness and has not been resolved through what has been reported in the prior art, is that in the places where the farms are, electricity, water and air are extremely scarce or nil; therefore a portable power plant or special arrangements to lower the energy of primary lines of 440V to the necessary voltage are required, and also the equipment require a controlled voltage and power quality, because electrical and electronic devices and mechanisms may suffer damage. Also, usually are required tankers that provide the amount of water needed, which can be up to 20 m3 per house, wasting a huge amount of liquid and making necessary the tanker to carry water from a well to the farm house, which means walking distances may be up to 5 km. As for the air, it is a service that virtually is not used in agribusiness, so to solve its supply is required a compressor and thus the complexity of electricity is the same.

Moreover for the generation of high-quality foam, it is necessary to have a high air and/or water pressure; however, such high pressure sources often cause security problems.

The prior art has revealed a very low foam generation capacity, which is due to the complexity in the technology to make a mixture of rich and dry foam and its movement to dose at high speed. This entails that in agriculture, derived from large areas, cleaning times become very extensive and hardly productive, because a limited time for cleaning process cycles is available, which would make unhelpful any technology.

Because in a mixture of liquid phase and gas phase, the flow rates are very different, transporting the mixture results in a “water hammer” originating a bad mixture of liquid phase and gas phase, and due to the high speed acquired by the gas, the equipment or pipes may result broken.

So today the processes for cleaning in agribusiness farms are very varied, from industrial pressure washers powered by electricity or combustion, centrifugal pumps that use a lot of water and almost do not use detergent or even a huge number of staff using brushes and containers of water with detergents scrubbing and scrubbing a large number m² daily to achieve the timely and proper completion of their industrial processes, so that a cleaning process that in addition to solving existing problems, also sanitize the area is necessary.

### SUMMARY OF THE INVENTION

Therefore an object of the present invention is to provide a system for cleaning and disinfection of agribusiness farms that overcomes existing drawbacks through a system comprising an autonomous mobile unit including: 1) a unit that produces large quantities of foam for detergent application, at least 40 l/min, which in turn generates between 4- and 6-fold said volume of foam, depending on how rich and dry is it desired, dosing between 150 1/min and 300 l/min of dry foam; II) a pressure washer unit that allows to connect from 2 to 8 hoses with special nozzles and works from 400 psi to 1500 psi of water pressure for the activities of pre-rinse and rinse.

Another object of the present invention is to provide a procedure for use and application of the mobile unit to increase the productivity of the cleaning and sanitization of agribusiness farms and reduce its mortality.

Also an object of the present invention is to improve the autonomous mobile cleaning unit from Utility Model Application No. MX/u/2012/000387 and PCT Patent Application No. PCT/MX2013/000104 by the same inventors and applicant that of the present invention, by incorporating a pressure water sub-unit for considering 2-8 hoses under the same principle of autonomy regarding energy, water and air, so that the optimal balance of energy and air to operate the various pumping equipment, pressure water and foam generation is achieved, thus having all the necessary resources in a space with the dimensions of a 2×5 meter trailer. Energy systems are based on gasoline or diesel motors, with the ability to be adapted to be powered by batteries and solar cells.

A further object of the present invention is to provide a cleaning agribusiness process comprising: 1) a pre-rinse with water at a pressure of between 400 PSI to 1500 PSI for all the surfaces of the houses(floors, ceilings, walls, structures, among others), equipment (feeders, drinkers, nipples, breeders, fans, among others) and curtains; with the aim to remove as much dirt as possible and moistening surfaces so that the detergent has better chemical action against dust; (2) the application in the form of foam, of a specialized veterinary/livestock use detergent either acidic, neutral or alkaline nature of high foam, in order that, being in the form of foam, the detergent allows greater contact time and thus chemical action is more effective, eliminating microorganisms and dirt from surfaces; (3) the final rinse with water at a pressure of between 400 PSI to 1500 PSI of all surfaces of the houses, including equipment and curtains, with the level of detail required to remove dirt from surfaces and equipment by pressure, starting with the ceilings and structures, then equipment, curtains, and finally floors and; (4) disinfection of surfaces either by fogging or spray with a sanitizer for veterinarian/livestock use. A further object of the present invention is to provide a cleaning and sanitizing system that improves the mobile unit of Utility Model Application No. MX/u/2012/000387 and PCT Patent Application No. PCT/MX2013/000104 owned by the applicant of the present invention, performing the cleaning of the agribusiness at the rate of 1200 m²/hr instead of 750 m²/hr through the cleaning process that includes the sub-unit for pre-rinse and rinse in the same unit, so that through the proposed cleaning procedure the cleaning is performed in an agribusiness standard installation of 1750 m² over a period of 1.4 hrs. allowing to cover an even larger number of m per day and/or reduce the number of operating personnel.

Another object of the invention is to provide a standalone mobile foam-producing unit for cleaning that improves the quality of the same, where the microbiological results prove the reduction in the aerobic mesophillic and fecal coliforms counts (as indicator organisms) with the simple use of the equipment and cleaning process of the present invention and consequently improves the level and quality of life of animals, reducing the use of vaccines, drugs and reduced mortality by up to 40%

### BRIEF DESCRIPTION OF THE FIGURES OF THE INVENTION

FIG. 1 is a perspective view of the cleaning and sanitizing system of the present invention.

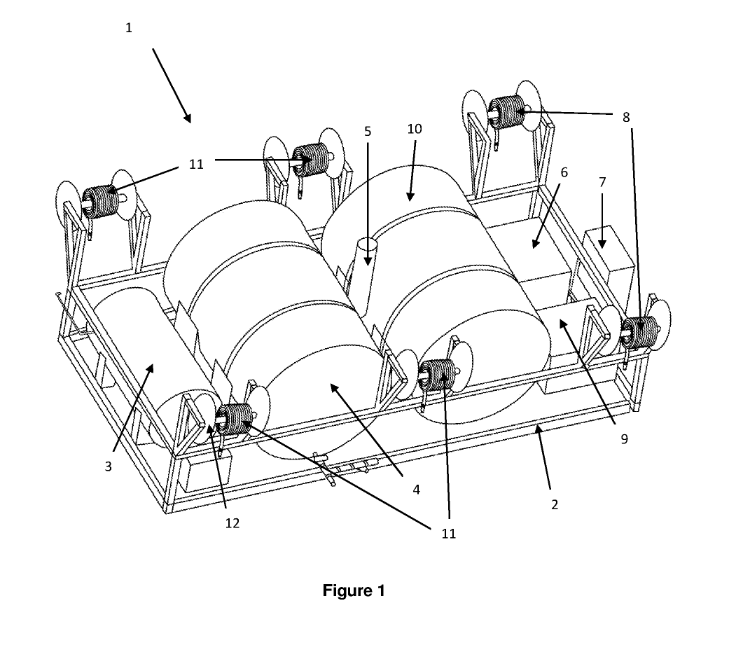

FIG. 2 is a plan view of the cleaning and sanitizing system of the present invention.

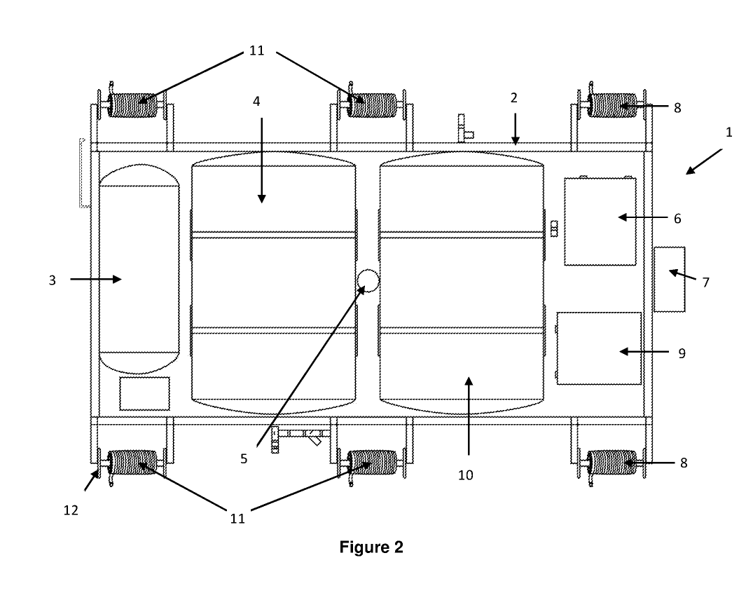

FIG. 3 is a side view of the cleaning and sanitizing system of the present invention.

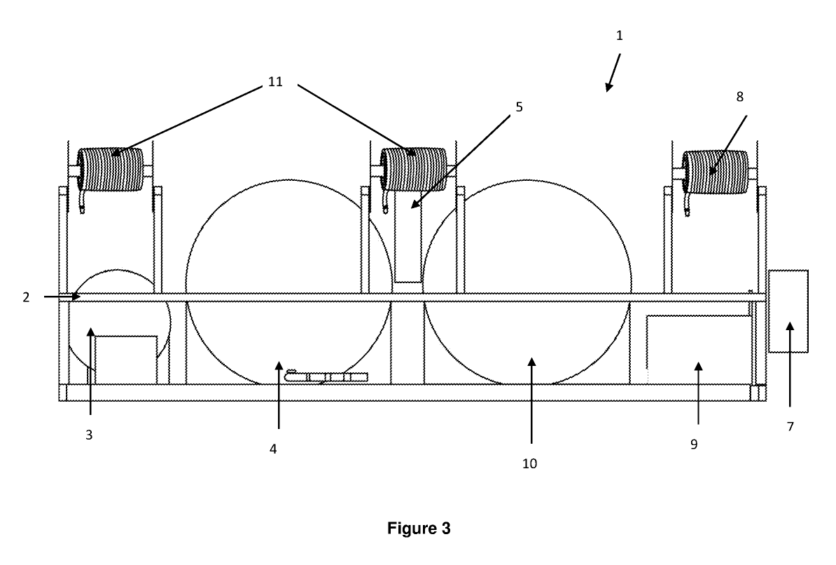

### DETAILED DESCRIPTION OF THE INVENTION

The present invention is described below with reference to the accompanying figures, in which reference numerals used refer to the same components in the different views.

The cleaning and sanitizing system of agro-industrial farms of the present invention comprises: 1) A cleaning standalone mobile unit 1 with two sub-units: one for foaming and other for water pressure,

The standalone mobile cleaning unit 1 comprises:

a) a trailer 2;

b) A foaming sub-unit comprising:

a. a gasoline-driven compressor 3;

b. a container tank 4 with a capacity of at least 1000 L,

c. Automatic detergent dosing system 5 disposed at the top for dispensing detergent to the desired concentration to at least 40 l/min;

d. a pump 6 either centrifugal or positive displacement;

e. a foam generator device 7 to generate foam through the compressed air, the detergent solution and a foaming cabin with a fluidized bed, connected to the dosing hoses 8, either 2 to 4 hoses, dosing between 150 l/min and 300 l/min of dry foam;

c) A pressure water unit comprising:

a. A positive displacement pump 9, either gasoline/diesel-driven or with connection to the driveshaft of the driving force of a tractor, to work with water pressure between 400 PSI and 1500 PSI

b. A container tank 10 with a capacity of at least 1000 L,

c. A head to operate from 2 to 8 high-pressure hoses 11 and;

d) Reels 12 for storing unused hoses.

The cleaning and sanitizing process of the present invention comprises the steps of:

a) Once the depopulation of the house/production house (batch output) is performed, the activities of removal of treated manure are made, and dry-cleaning is carried out, proceeding to perform a pre-rinse with water pressure from 400 PSI to 1500 PSI of all surfaces of the houses (floors, ceilings, walls, structures, among others), equipment (feeders, drinkers, nipples, breeders, fans, among others) and curtains, using the pressure water sub-unit in order to remove as much dirt and microorganisms as possible, and moisten surfaces for a better chemical action of the detergent against dirt and microorganisms.

b) Apply, in the form of foam, using the foaming sub-unit, a detergent for veterinary livestock use either acidic, neutral or alkaline of high foam. The purpose of applying the detergent in the form of a foam is to allow a greater contact time with the surfaces to be cleaned, and therefore achieving a more effective chemical action, removing dirt and microorganisms from surfaces.

c) Perform a final rinse using the pressure water sub-unit with water pressure between 400 psi to 1500 psi of all surfaces of the houses, including equipment and curtains, with the level of detail required to remove dirt from surfaces, starting with the ceilings and structures, then the equipment, curtains and finally floors.

d) Perform disinfection of surfaces either by thermofogging or fogging with a sanitizer for veterinary/livestock use.

The present innovation provides a novel system with autonomy in energy, water and air, so that the proper balance of energy and air to operate the various pumping equipment and foam generation is accomplished, having all the necessary resources for cleaning and sanitizing within a space with the dimensions of a 2×5 m trailer. Energy systems are gasoline or diesel-driven, with the ability to be adapted to be powered by batteries and solar cells.

The system of the present invention allows the cleaning and sanitizing at the rate of 1200 m²/hr, which would make a standard installation of 1750 m² to be cleaned over a period of 1,417 hours allowing to cover an even larger number of m² per day or reduce the number of operating personnel.

Also, use of the cleaning system according to the present invention has shown to significantly reduce mortality of farm animals, and thus the costs of vaccines and drugs, helping improving the total production costs in an industry with very low margins, achieving extreme cases going from 25% mortality to less than 5%.

A microbiological analysis of areas where cleaning and sanitization was performed by standard cleaning processes with any of the equipment or existing techniques showed the following data:

Floors 2,200 CFU/cm² mesophillic aerobic and 180 CFU/cm² fecal coliforms;

In the trough 1800 CFU/cm² mesophillic aerobic and 500 CFU/cm² fecal coliforms.

With the process and cleaning and sanitizing system of the present invention, results in the same sampling points were:

Floors 370 CFU/cm² mesophillic aerobic, less than 10 CFU/cm² fecal coliforms, 1 CFU/cm² fungal, and 8 CFU/cm² yeast; In the trough 380 CFU/cm² aerobic mesophillic, 60 CFU/cm² fecal coliforms, 1 CFU/cm² fungal, and 14 CFU/cm² yeast.

From the above results, a 1-log reduction in aerobic mesophillic bacteria, and until 2-log reduction in fecal coliforms are observed, using the process and cleaning and sanitization system of the present invention, so that it is shown as cleaning and sanitization with the process and cleaning and sanitizing system of the present invention using the foam generated is highly effective in removal of dirt and microorganisms.

The present invention has been described according to a preferred embodiment and will be apparent to one skilled in the art that modifications may be made thereto without departing from the spirit and scope thereof.

1. A cleaning and sanitization system for agribusiness farms comprising:
a stand-alone cleaning unit comprising a foaming sub-unit having a compressor, a container tank, an automatic detergent dosage system, a pump, and a foam generator device or foaming cabin through compressed air, and 2-4 hoses for foam application, being connected these elements in such a way that the dispenser automatically dispenses detergent at a rate of at least 40 l/min, so that when mixed with water and compressed air, it provides a dry foam that it is expelled through dispensing hoses, dosing between 150 l/min and 300 l/min dry foam;
a pressure water sub-unit consisting of a positive displacement pump, a container tank, and 2-8 high-pressure hoses connected through a head, to expel water under pressure and
reels for storing unused hoses.

2. The system according to claim 1, wherein the container tank of the foaming sub-unit and the container tank of the water pressure sub-unit, have a capacity of at least 1000 liters.

3. The system according to claim 1, wherein the positive displacement pump is connected to a gasoline/diesel motor or driveshaft of driving force of a tractor.

4. The system according to claim 1, wherein the positive displacement pump allows the water of the pressure water sub-unit to have a pressure of 400-1500 PSI.

5. The system according to claim 1, wherein the standalone unit includes a trailer upon which the foaming sub-unit and the water pressure sub-unit are mounted.

6. A process for cleaning and sanitizing agribusiness farms comprising the steps of:
once the depopulation of the production house/unit, the removal of treated manure, and dry-cleaning are carried out, a pre-rinse of all surfaces, equipment and curtains is performed with water at a pressure of between 400 PSI to 1500 PSI, using a pressure water sub-unit to remove as much dirt and microorganisms as possible, and at the same time wetting surfaces so that the detergent has better chemical action against dust and harmful bacteria;
apply a detergent for veterinary/livestock use in the form of foam, using a foaming sub-unit, allowing the foamed detergent to be in contact with the surface to be cleaned long enough to make the chemical action more effective, removing dirt and microorganisms from surfaces;
perform, using a pressure water sub-unit, a final rinse with water at a pressure of between 400 PSI-1500 PSI in all surfaces, with the level of detail required to remove dirt from surfaces by pressure; and
carry out disinfection of surfaces either by thermofogging or fogging with a sanitizer for veterinarian/livestock use.

7. The process according to claim 6, wherein the cleaning and sanitization is performed at a rate of 1200 m²/hr.

8. The process according to claim 6, wherein the cleaning and sanitization reduce the presence of aerobic mesophillic bacteria and fecal coliforms, as well as reference microorganisms.
